(19) United States
(12) Patent Application Publication
(10) Pub. No.: US 2021/0369374 A1
(43) Pub. Date: Dec. 2, 2021

(54) MASTER CONTROLLER ASSEMBLY FOR A ROBOTIC SURGERY SYSTEM, PARTICULARLY FOR MICROSURGERY

(71) Applicant: MEDICAL MICROINSTRUMENTS S.P.A., Calci, Pisa (IT)

(72) Inventors: Massimiliano SIMI, Calci, Pisa (IT); Giuseppe Maria PRISCO, Calci, Pisa (IT)

(21) Appl. No.: 17/056,355

(22) PCT Filed: May 17, 2019
(86) PCT No.: PCT/IB2019/054095

§ 371 Date: Nov. 17, 2020

(30) Foreign Application Priority Data
May 17, 2018 (IT) 102018000005469
Aug. 24, 2018 (IT) 102018000008179
Aug. 30, 2018 (IT) 102018000008251

(51) Int. Cl. A61B 34/00; A61B 34/37; B25J 3/00; B25J 9/00

(52) U.S. Cl. CPC A61B 34/74; A61B 34/37; B25J 9/0009; B25J 3/00; A61B 34/73

(57) ABSTRACT

A master controller assembly for a robotic surgery system has a slave robot assembly, a slave surgical instrument and a control unit. The master controller assembly has a master input tool and a convex manipulandum surface to be hand-held by surgeon's fingers. The master input tool is mechanically unconstrained from the slave robot assembly, naturally movable, rotatable and spinnable by the surgeon. The master input tool includes first and second elongated elements having respectively a first element elongated body and a second element elongated body. A tool joint connects and articulates the first and second element elongated bodies. A sensing assembly detects mutual position of the first and second element elongated bodies, so that a gripping pressure action exerted by the surgeon's fingers on the master input tool moves the first and second element elongated bodies closer and determines a paired grip motion of a surgical grip device.

### FIELD OF THE INVENTION

It is an object of the present invention a master controller assembly for a robotic surgery system.

Moreover, the present invention relates to a robotic surgery system.

In particular, said robotic surgery system is suitable for microsurgery.

### BACKGROUND

Robotic surgical assemblies comprising a master interface and a slave surgical tool are generally known in the field. Specifically, robotic surgical assemblies of the known type comprise a master control station able to control the motion of a slave surgical end-effector, as shown for example in document U.S. Pat. No. 5,876,325. This document disclose a non-portable, robot-hung articulated appendices, which are hung to a beam fixedly constrained to the master control station, said appendices comprise master tools to control the slave surgical end-effector operating on a patient anatomy.

Similar non-portable robot-held master tool solutions are shown, for example, in documents U.S. Pat. Nos. 6,063,095, 6,424,885 and 6,594,552, wherein the appendix of the master control station acting as master tool to control the slave surgical end-effector comprises an appendix body rigidly constrained to the master control station. The transmission of motion to the slave end-effector is based on the detection of mechanical stress induced by urging the appendix body of the master control station in various spatial directions. Such an appendix body can be associated to a pair of opposite fins, each of said fins being constrained on one of its end to the appendix body in such way to form a cantilevered fins, suitable for receiving a manual command directed to activate the grip degree-of-freedom of the slave end-effector.

However, non-portable, robot-hung or robot-held master tool solutions of the types described above exhibit some drawbacks. The provision of such control appendix mechanically constrained to the master control station of the robotic surgical assembly strongly limits the natural freedom of motion of the surgeon during surgery and forces the surgeon to operate in a predefined location from which the master control station, and particularly the control appendix attached thereto, is easily reachable. The discomfort for the surgeon is still enhanced due to the inability to real-time adjust during surgery the location, for example in terms of height from the soil, of such an appendix. That leads the surgeon to an untimely tiredness during surgery and to early focus loss.

Often, surgeons have been trained for years to properly handle surgical tools suitable for operating directly on a patient anatomy. Surgical tools are generally portable tools and comprise a tool handle, suitable to be hand-held and manipulated by the surgeon, said handle being mechanically directly connected to a tool tip, suitable for operating on a patient anatomy. Some examples of traditional ophthalmic surgery surgical tools are shown in documents U.S. Pat. No. 5,634,918 and WO-2012-064361. Such traditional surgery tools make the surgeon sure to know when the tool tip is free from touching the patient anatomy, in this way allowing the surgeon to safely (i.e. without transmitting actions on the patient anatomy) roll the tool handle between the fingers around the longitudinal axis of the tool handle, a gestural stress-reducing need rather common among surgeons for example useful for relax the hand muscles during surgery and to prevent muscular spasms.

Robotic microsurgery, instead, forces the surgeon to use master tools to control the motion of an associated slave end-effector operating on a patient anatomy, and usually said master tools limit the comfort of the surgeon during surgery and often force the surgeon to an additional period of training for properly using the master tool to control the slave end-effector. The additional training period can be even lengthen if the of the shape and functionalities of the master tool are alien from a traditional surgical tool.

Wearable master tool have been provided, as disclosed for example in document U.S. Pat. No. 8,996,173, wherein a pair of rings are designed to be fit on surgeon's finger and wired to the robotic slave assembly. A codified gesture set of the surgeon's finger triggers a predefined slave end-effector action on the patient anatomy. Obviously, this solution requires a very long training to the surgeon for properly managing such a wearable master rings, in order to avoid to transmit unintended commands to the slave end-effector. Unintended command transmission to the slave should be avoided for patient safety reasons. Also documents DE-102014006264 and DE-102010009065 shows a wearable master tool.

To overcome the deficiencies of known solution described above and in order to provide an hand-held manipulandum (i.e. from the Latin: “something to be manipulated”) master tool having a shape which is familiar for most surgeons, documents WO-2017-064303 and WO-2017-064306, in the name of the same Applicant, show a master tool device which substantially replicates the appearance of a traditional surgery tweezers. Such master tool device comprises a pair of flexible strips of metal welded together in one of their ends to form a tweezers-like master input device. Suitably located sensors help the magnetic pad to track the motion of the tweezers and detect when the tweezers close, in order to mimic an object grasp and to transmit the detected motion to the slave surgical end-effector.

Although satisfactory to improve the surgeon's comfort during surgery, this type of solution is prone to drawbacks. In particular, such flexible metal strips forming a tweezers-like device force a non-linear motion of the sensors placed on the free end of the metal strips, thus the detection of the manually-induced tweezers closing motion, for mimicking a sort of object grasp, often leads to measurement uncertainty and low sensing resolution. Mechanical vibrations arising in each metal strip during its elastic bending generate noise detected by the tracking pad. That could result in an unsatisfactory motion response of the slave end-effector that could even lead to serious complications in the patient body after surgery. Moreover, the tracking pad is suitable for generating a tracking magnetic field only from one side of the pad, forcing the surgeon not to move the manipulandum hand-held master tool on the back side of the pad, where the motion cannot be tracked, thus a command signal cannot be transmitted to the end-effector.

Furthermore, documents US-2013-0035697, WO-2014-151621 and US-2015-038981 disclose a portable, hand-held master input tool manipulatable by a surgeon while moving in various locations of the operating arena. This solution exploits video-camera tracking of suitably designed balls that protrudes cantilevered from the portable hand-held master tool body. In other words, a set of three non-symmetric balls mounted on the master tool can be tracked by a camera apparatus provided on-robot to determine the position and orientation of the master input tool with the aim to transmit a command signal to the slave surgical end-effector.

Although satisfactory under some points of view, this solution is prone to drawbacks. As the visual-cues-based tracking system allows the surgeon to operate while moving in various locations of the operating arena, at the same time force the robot to have a powerful control system and can result in an unwanted delay of transmission of motion to the slave end-effector, resulting in a discomfort for the surgeon.

The need is felt to provide a master tool solution for robotic surgery able to overcome the drawbacks cited with reference to the prior art.

The need is felt to provide a master tool for robotic surgery suitable for improving the surgeon's comfort and at the same time able to provide a high sensing accuracy.

The need is felt to provide a master tool for robotic surgery able to reduce to a minimum the length of the surgeon training.

The need is felt to provide a master tool for robotic surgery suitable for avoiding the transmission of unwanted command signal to the slave end-effector.

The need is felt to provide a master tool for robotic surgery devoid of mechanical constraint to the master control station or to the slave robot.

### SOLUTION

It is a scope of the present invention to overcome the drawbacks mentioned with reference to the prior art.

These and other scopes are achieved by a master controller assembly according to claim 1, as well as a robotic surgery system according to claim 18.

Some preferred embodiments are the subject of dependent claims.

According to an aspect of the invention, a master controller assembly comprises at least one master input tool and at least one sensing assembly, wherein said master input tool comprising at least one manipulandum surface, designed to be hand-held by the surgeon's fingers and is mechanically unconstrained from said slave robot assembly, in such way that said master input tool being naturally movable, rotatable and spinnable by a surgeon. Said at least one manipulandum surface is a convex surface, so that said master input tool (106) can be rolled between surgeon's fingers around a tool longitudinal axis. Thereby the surgeon comfort during surgery is preserved. Said master input tool comprises a first elongated element having an first element elongated body, wherein said first element elongated body is a rigid body, and a second elongated element having an second element elongated body, wherein said second element elongated body is a rigid body, and a tool joint connecting and articulating said first element elongated body and said second element elongated body, providing a single degree of freedom of motion between said first element elongated body and said second element elongated body.

The at least one sensing assembly detects at least the mutual position of said first element elongated body and said second element elongated body so that a gripping pressure action exerted by the surgeon's fingers on said master input tool moving said first element elongated body and said second element elongated body close one another other determines a paired grip motion of said surgical grip device.

Said sensing assembly may comprise a pair of sensors received in respective slots of the master input tool body. Each slot may be mechanically shaped to be compatible with only one of the sensor.

Each sensor of the sensing assembly may be encapsulated by a sterile barrier, such as a plastic bag or box, to allow the re-use of the sensors. The master input tool body may be disposable.

### FIGURES

Further characteristics and advantages of the master controller assembly and of the robotic surgery system according to the invention will appear from the description reported below of preferred embodiments, which are given as examples and are not meant to be limiting, which makes reference to the attached drawings, in which:

FIGS. 1 and 1b is are perspective views showing a robotic surgery system, according to some embodiments;

FIGS. 2 to 5 are perspective views showing master controller assembly, according to some embodiments, hand-held by surgeon;

FIG. 6 is a perspective view showing master controller assembly, according to an embodiment;

FIGS. 7 and 8 are perspective views of master controller assembly, according to some embodiments, showing as separated pieces a sensing assembly and a master input tool;

FIG. 9 is a perspective exploded view of a master input tool, according to an embodiment;

FIG. 10 is a longitudinal cross-section of master input tool realized along the cut plane indicated with X-X-X-X in FIG. 8;

FIG. 11 is a perspective view of master controller assembly, according to an embodiment;

FIGS. 12 and 13 are longitudinal cross-sections of master input tool in closed positons, according to an embodiment;

FIG. 14 is a longitudinal cross-section of trigger of master input tool, according to an embodiment;

FIG. 15 is a longitudinal cross-section of tool joint of master input tool, according to an embodiment;

FIG. 16 is a perspective view of master controller assembly, according to an embodiment, wherein sensing assembly is shown as separate parts in respect of the master input tool;

FIG. 17 is a perspective view of master controller assembly, according to an embodiment;

FIG. 18 is a perspective view of master controller assembly, according to an embodiment, being hand-held by surgeon;

FIG. 19 is a sketch in perspective view showing robotic surgery system comprising paired master controller assembly and slave surgical grip device, according to an embodiment;

FIG. 20 is a perspective view showing master controller assembly, according to an embodiment;

FIG. 21 is a perspective view of robotic surgery system, according to an embodiment;

FIGS. 22 and 23 are block diagrams showing a robotic surgery assembly, according to some embodiments.

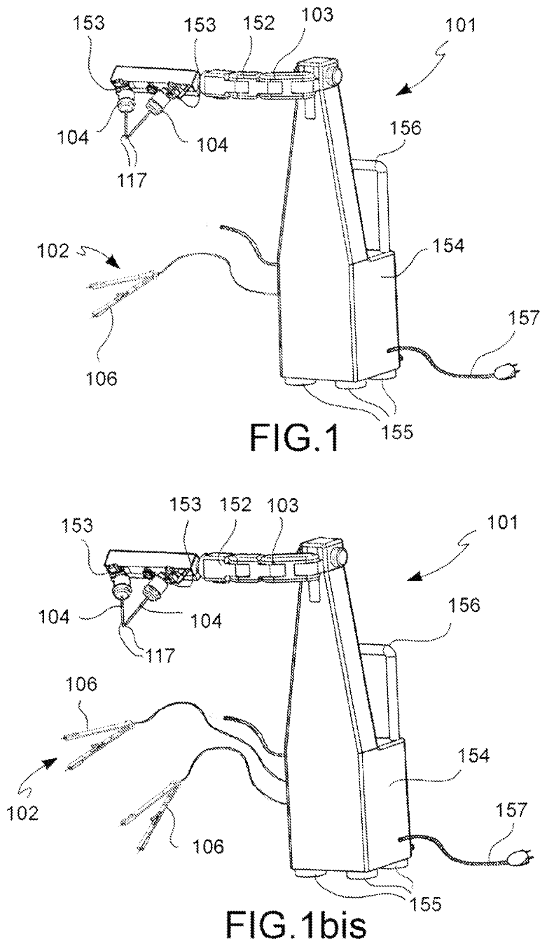

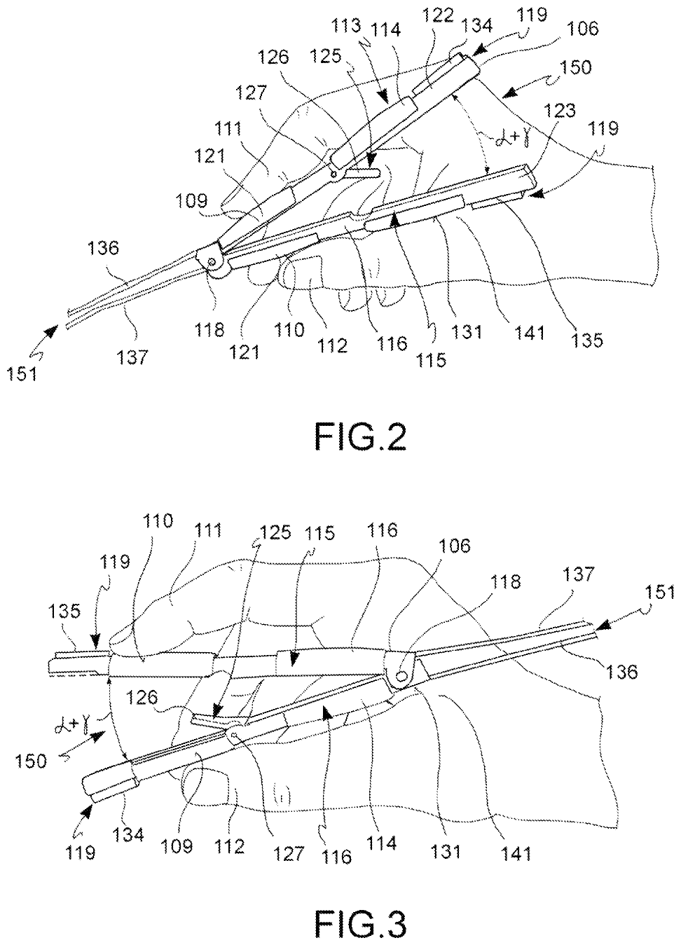

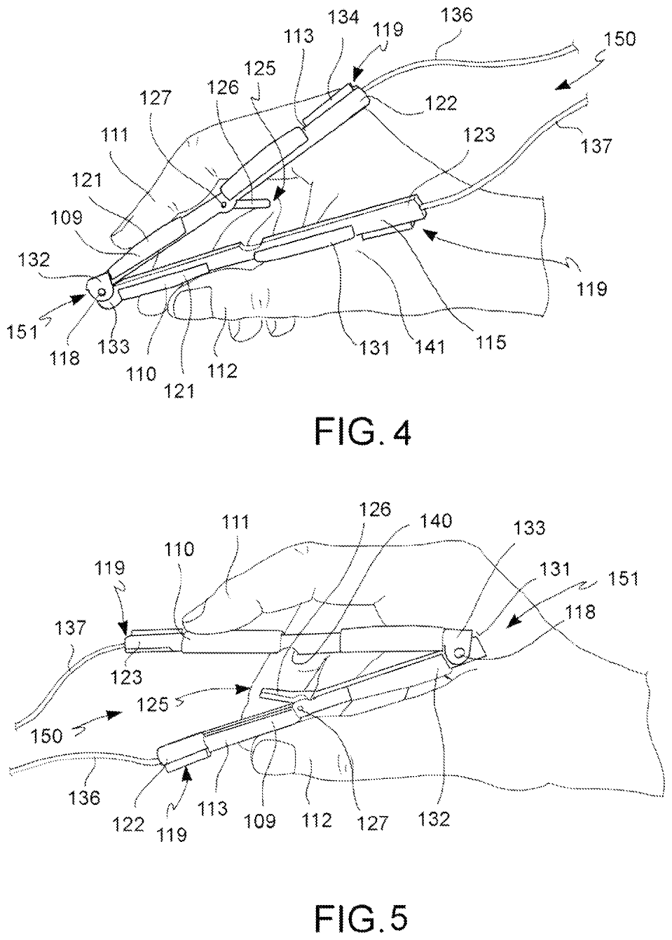

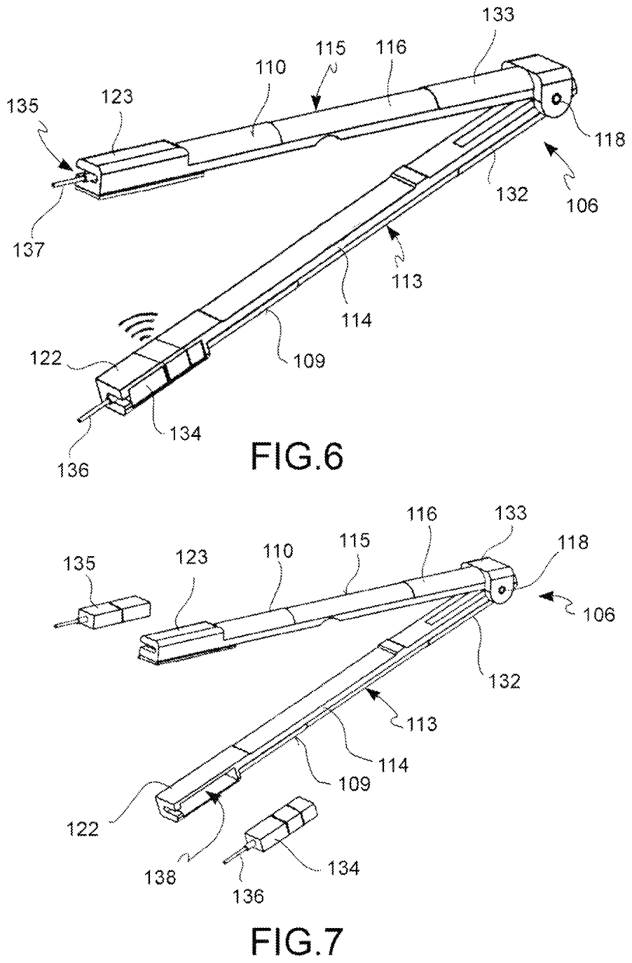

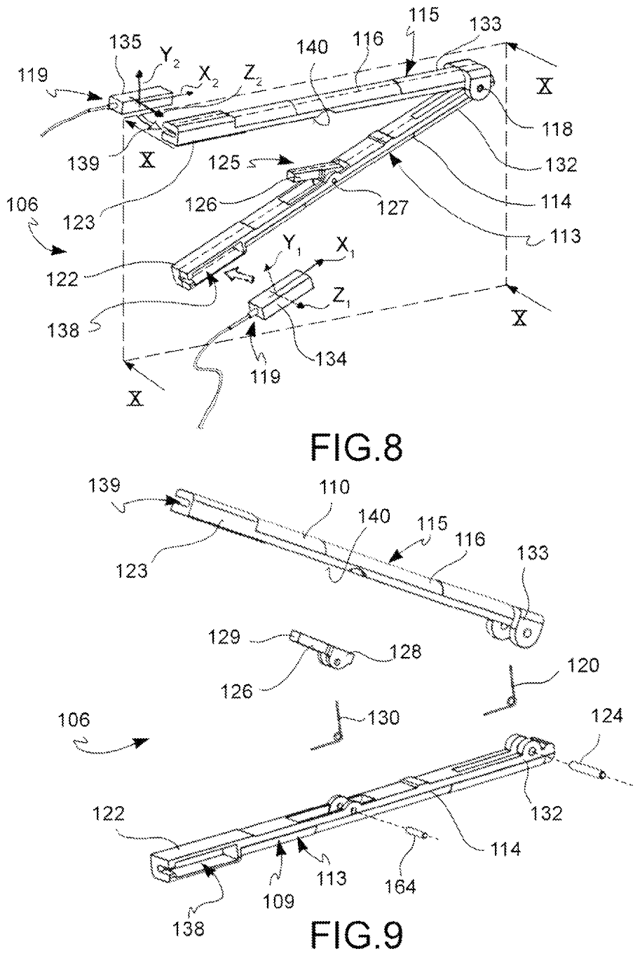

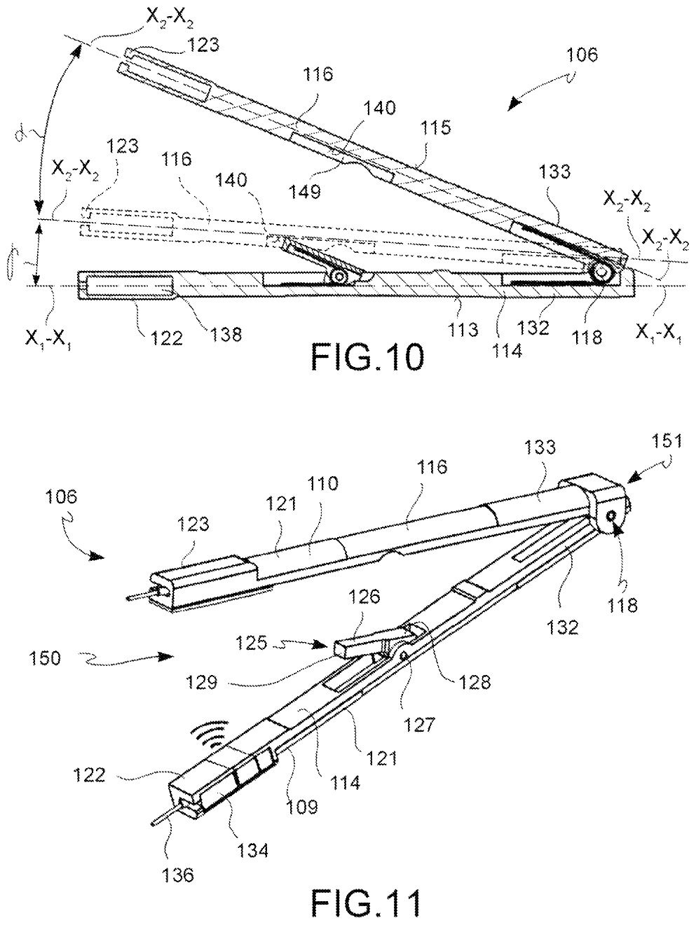

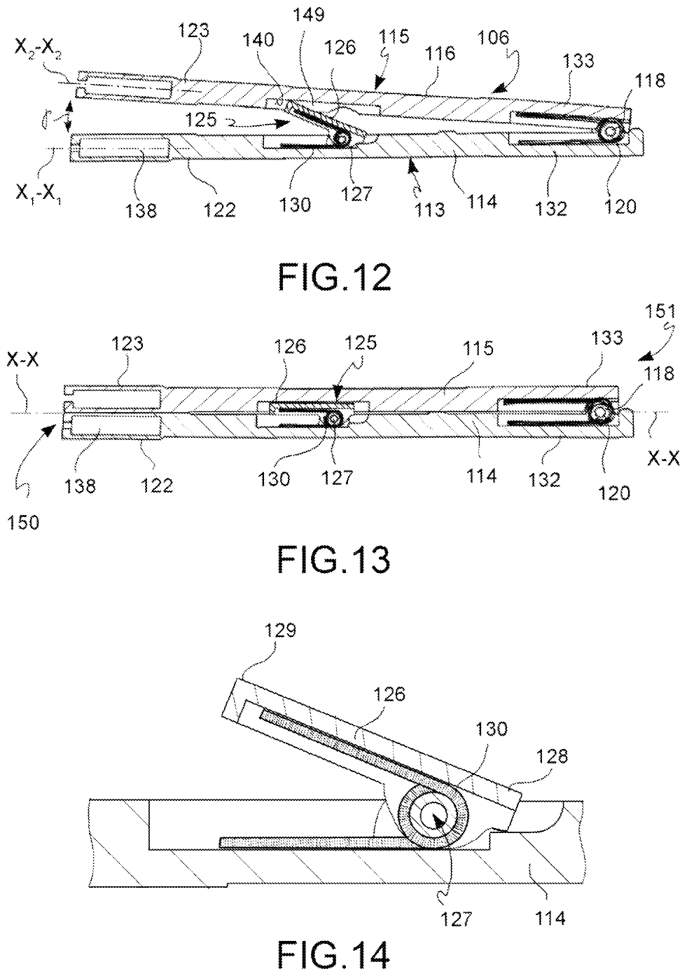

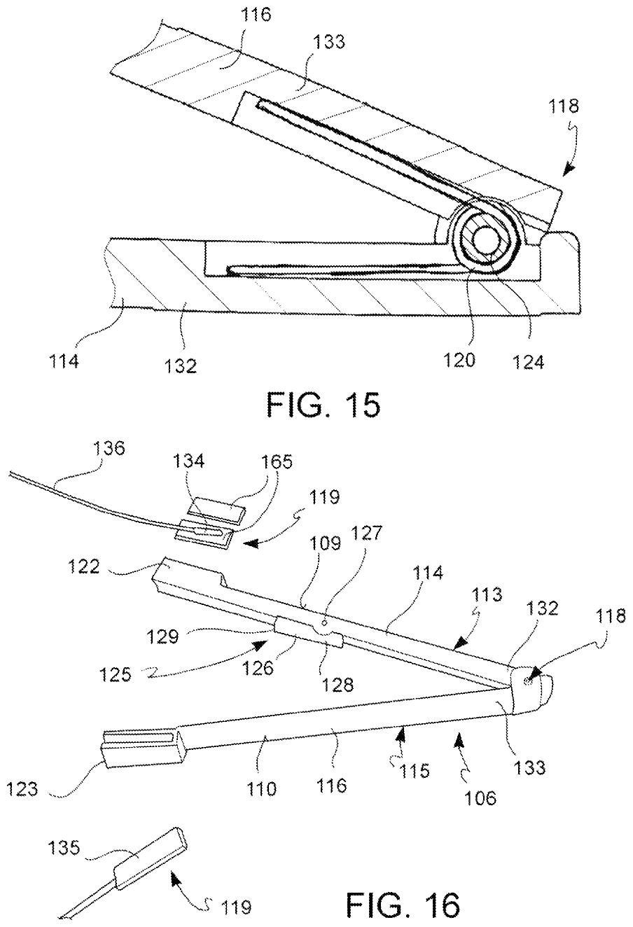

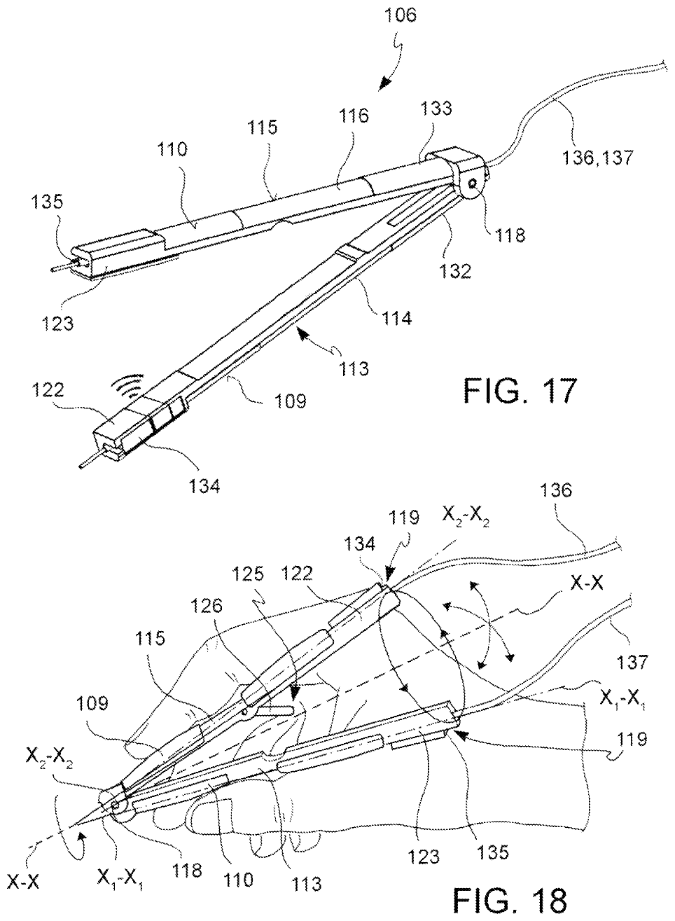

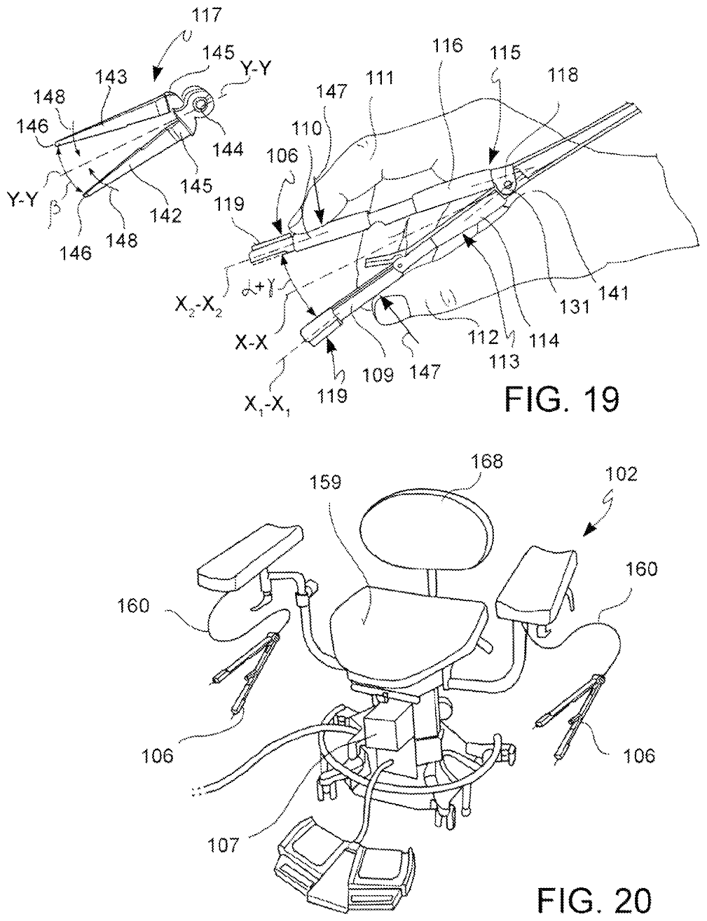

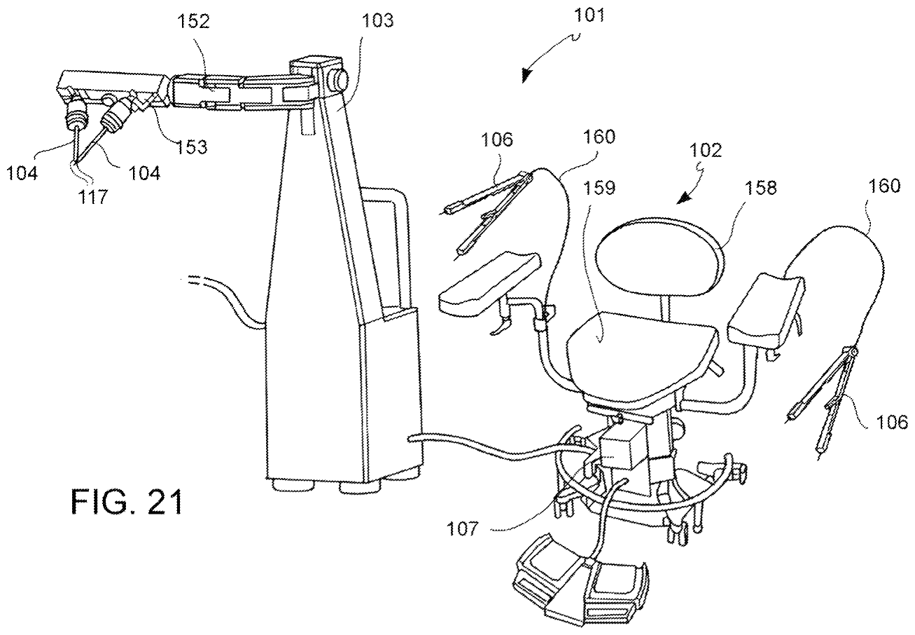

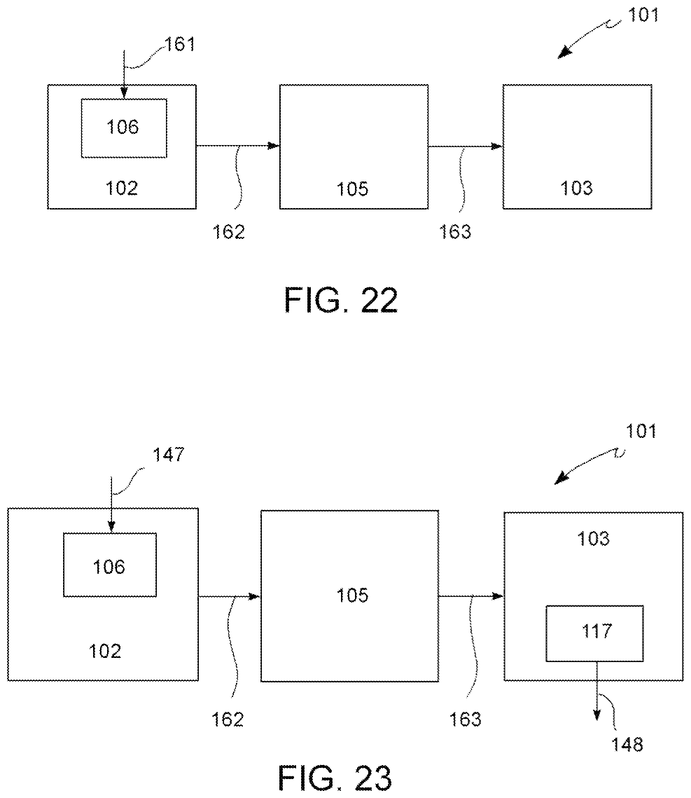

### DETAILED DESCRIPTION OF SOME EMBODIMENTS

According to a general embodiment, a robotic surgery system 101 comprises at least one master controller assembly 102, suitable to detect a manual command 161, and at least one slave robot assembly 103, comprising a slave surgical instrument 104 designed to operate on a patient anatomy.

Said slave surgical instrument 104 comprises at least one surgical grip device 117 providing the slave surgical instrument 104 with a grip degree-of-freedom of motion.

According to an embodiment, said surgical grip device 117 comprises a first elongated element of surgical grip device 142 and a second elongated element of surgical grip device 143, said first elongated element of surgical grip device 142 and said second elongated element of surgical grip device 143 being articulated in respect to one another forming a surgical grip joint 144, preferably said surgical grip joint 144 being a pin joint. Preferably, each of said first elongated element of surgical grip device 142 and said second elongated element of surgical grip device 143 comprises a joint portion of surgical grip device 145, forming at least a portion of said surgical grip joint 144, and a cantilevered free end of surgical grip device 146.

According to a preferred embodiment, said robotic surgery system 101 comprises a control unit 105, suitable for receiving a first command signal 162 containing information about said manual command 161 and to transmit a second command signal 163 containing information about said manual command 161 to the slave robot assembly 103 in order to actuate said slave surgical instrument 104.

According to a preferred embodiment, said master controller assembly 102 is paired along a master-slave pair to said slave surgical instrument 104. According to a preferred embodiment, said master controller assembly 102 and said slave surgical instrument 104 form, trough said control unit 105, a master-slave pair.

Said master controller assembly 102 comprises at least one portable hand-held master input tool body 106 (or master input tool 106), suitable to be hand-held and manipulated by a surgeon from various locations of an operating arena during surgery. In this way, said master controller assembly 102 is provided with portability, for example during surgery within said operating arena. Preferably, said portable hand-held master input tool 106 is hand-held and manipulated by a surgeon from various locations of an operating arena during surgery. Preferably, said master input tool 106 receive said manual command.

According to a preferred embodiment, the term “portable” referred to said master input tool indicates that the master input tool is capable to be carried or moved about, for example by the surgeon during surgery.

According to a preferred embodiment, the term “hand-held” referred to said master input tool indicates that the master input tool is designed to be operated while held in a hand, for example the surgeon's hand.

According to a preferred embodiment, the term “operating arena” refers to a portion of space at least partially surrounding a patient anatomy. Preferably, within the operating arena are comprised various locations beside the patient anatomy.

According to a preferred embodiment, the term “manipulated” referred to said master input tool indicates that the master input tool can be treated or operated with or as if with hands.

According to a preferred embodiment, said master input tool 106 is paired along a master-slave pair to said slave surgical instrument 104. According to a preferred embodiment, said master input tool 106 and said slave surgical instrument 104 form, trough said control unit 105, a master-slave pair.

Said master controller assembly 102 is operatively connected to said slave robot assembly 103. According to an embodiment, said master controller assembly 102 is connected to said slave robotic assembly 103 by means of electromagnetic communication.

Said master input tool 106 comprise at least one manipulandum surface 109, 110, designed to be hand-held by the surgeon's fingers 111, 112. In this way, the portability of the master input tool 106 is enhanced.

Said master input tool 106 is mechanically unconstrained from said slave robot assembly 103, in such way that said master input tool 106 is, preferably naturally, movable, rotatable and spinnable by a surgeon.

Said master input tool 106 is mechanically ungrounded.

According to an embodiment, said master input tool is unsuitable for providing force feedback.

Said at least one manipulandum surface 109, 110 is a convex surface, so that said master input tool 106 can be rolled between surgeon's fingers 111, 112 around a tool longitudinal axis X-X.

According to a preferred embodiment, said master input tool 106 comprises a first elongated element 113 having a first element elongated body 114, wherein said first element elongated body 114 is a rigid body. According to a preferred embodiment, the terminology “rigid body” means that such a body is deficient or devoid of flexibility. According to an embodiment, the terminology “rigid body” means that such a body is unable to provide elastically flexural deformation when in operative conditions.

According to an embodiment, said first element elongated body 114 defines a first element direction X1-X1, substantially coincident with the axis of longitudinal development of said first element elongated body 114.

According to a preferred embodiment, said master input tool 106 comprises a second elongated element 115 having a second element elongated body 116, wherein said second element elongated body 116 is a rigid body.

According to an embodiment, said second element elongated body 116 defines a second element direction X2-X2, substantially coincident with the axis of longitudinal development of said second element elongated body 116.

According to a preferred embodiment, said master input tool 106 comprises a tool joint 118 connecting and articulating said first element elongated body 114 and said second element elongated body 116, providing a single degree of freedom of motion between said first element elongated body 114 and said second element elongated body 116.

According to an embodiment, said single degree of freedom of motion between said first element elongated body 114 and said second element elongated body 116 lies in a predefined plane.

According to a preferred embodiment, said master controller assembly 102 comprises at least one sensing assembly 119 detecting at least the mutual position, preferably the mutual position and the relative orientation, of said first element elongated body 114 and said second element elongated body 116. In this way, a gripping pressure action 147 exerted by the surgeon's fingers 111, 112 on said master input tool 106 moving said first element elongated body 114 and said second element elongated body 116 close one another other, determines a paired slave grip motion 148 of said surgical grip device 117.

Thanks to the fact that said first element elongated body 114 and said second element elongated body 116 are both rigid bodies, the sensing resolution of said sensing assembly 119 is improved in respect of known solutions.

According to an embodiment, said manual command 161 comprises said gripping pressure action 147.

According to an embodiment, said paired slave grip motion 148 moves said first elongated element of surgical grip device 142 and a second elongated element of surgical grip device 143 close to one another.

According to an embodiment, said gripping pressure action 147 is exerted by the surgeon's fingers 111, 112 on said at least one manipulandum surface 109, 110 of the master input tool 106.

According to an embodiment, said sensing assembly 119 comprises at least one capacitive incremental position sensor, for example a capacitive encoder.

According to an embodiment, said robotic surgery system 201, preferably said master controller assembly 102, comprises at least one field generator 107 generating a predefined field volume. According to a preferred embodiment, said at least one filed generator 107 generates a magnetic field.

According to an embodiment, said at least one sensing assembly 119 detects at least the position, preferably at least the position and the orientation, of said master input tool 106 within said predefined field volume.

According to an embodiment, said field generator 107 defines a reference zero point X0,Y0,Z0 integral with said field generator 107, and wherein said at least one sensing assembly 119 detecting the generated field local vector X1,Y1,Z1;X2,Y2,Z2, determines at least the position of said sensing assembly 119. In this way, sensing assembly 119 determines at least the position of said master tool assembly 106 integral with said sensing assembly 119 within said predefined field volume.

According to an embodiment, said master controller assembly 102 is operatively connected to said slave robot assembly 103 by means of a wired electric connection.

According to an embodiment, said master controller assembly 102 is operatively connected to said slave robot assembly 103 by means of a wireless connection.

According to an embodiment, said master input tool 106 is mechanically unconstrained from both the field generator 107 and the slave robot assembly 103, in such way that said master input tool 106 being naturally movable, rotatable and spinnable by a surgeon within said predefined filed volume.

According to an embodiment, at least one of said first element elongated body 114 and said second element elongated body 116 comprises said at least one manipulandum surface 109, 110.

According to a preferred embodiment, each of said first element elongated body 114 and said second element elongated body 116 comprises said at least one manipulandum surface 109, 110. In this way, said first element elongated body 114 comprises a first manipulandum surface 109, and said second element elongated body 116 comprises a second manipulandum surface 110.

According to an embodiment, said at least one manipulandum surface 109, 110 comprises a friction enhanced portion 121, suitable for improving the grip of surgeon's fingers 111, 112 thereon.

According to an embodiment, said at least one manipulandum surface 109, 110 is a portion of a cylindrical surface. In this way, the rollability of the master input tool 106 around a tool longitudinal axis X-X is enhanced.

According to an embodiment, said first manipulandum surface 109 and said second manipulandum surface 110 cooperate to form at least a portion of a cylindrical surface. In this way, the rollability of the master input tool 106 around a tool longitudinal axis X-X is enhanced.

According to an embodiment, said tool joint 118 is a hinge providing a single degree of freedom of motion of rotation between said first element elongated body 114 and said second element elongated body 116. In this way, said first element elongated body 114 and said second element elongated body 116 are movable in respect of one another of an angular movement.

According to an embodiment, a master gripping angle α+γ is defined as the angle between said first element elongated body 114 and said second element elongated body 116. According to an embodiment, the angle between said first element elongated body 114 and said second element elongated body 116 defines a master gripping angle α+γ.

According to an embodiment, said first element elongated body 114 and said second element elongated body 116 are movable in respect of one another of angular motion between at least one open position, wherein said master gripping angle α+γ is greater than a predefined grip threshold angle γ, and at least one closed position, wherein said master gripping angle α+γ is smaller than a predefined grip threshold angle γ.

According to a preferred embodiment, said master gripping angle α+γ is equal to or lower than 60 degrees, when said first element elongated body 114 and said second element elongated body 116 are in an open position. Preferably, said master gripping angle α+γ is equal to or lower than 45 degrees, when said first element elongated body 114 and said second element elongated body 116 are in an open position. said master gripping angle α+γ is equal to or lower than 35 degrees, when said first element elongated body 114 and said second element elongated body 116 are in an open position.

According to an embodiment, said tool joint 118 is a pin joint providing a single degree of freedom of motion, preferably of angular motion, of rotation between said first element elongated body 114 and said second element elongated body 116.

According to an embodiment, the tool longitudinal axis X-X is defined as being coincident with the bisector of said master gripping angle α+γ.

According to an embodiment, the tool longitudinal axis X-X is defined as the set of points that are equidistant from said first element direction X1-X1 and from said second element direction X2-X2.

According to an embodiment, the tool longitudinal axis X-X is defined as the axis of longitudinal development of said master input tool 106, when said first element elongated body 114 and said second element elongated body 116 are in a closed position.

According to an embodiment, said first element elongated body 114 comprises a first element joint portion 132, forming a portion of said tool joint 118, and a first element cantilevered portion 122, located opposite to said first element joint portion 132 along the first element direction X1-X1.

According to an embodiment, said second element elongated body 116 comprises a second element joint portion 133, forming a portion of said tool joint 118, and a second element cantilevered portion 123, located opposite to said second element joint proximal portion 132 along the second element direction X2-X2.

Preferably, said first element cantilevered portion 122 form a free end, and said second element cantilevered portion 123 form a free end.

According to an embodiment, the relative spatial position of said first element cantilevered portion 122 and said second element cantilevered portion 123 is rigidly determined by said master gripping angle α+γ width. In this way, the sensing resolution of said sensing assembly 119 is enhanced.

According to a preferred embodiment, said first element joint portion 132 and said second element joint portion 133 cooperate to form said tool joint 118.

According to an embodiment, said first element joint portion 132 and said second element joint portion 133 are constrained one another by means of a tool joint pin 124 to form said tool joint 118.

According to an embodiment, said first element cantilevered portion 122 and said second element cantilevered portion 123 are located at a predefined distance from said tool joint 118 along said first element elongated body 114 and said second element elongated body 116, respectively.

According to an embodiment, said sensing assembly 119 comprises at least one joint sensor, preferably an encoder, located within said tool joint 118.

According to an embodiment, said sensing assembly 119 comprises at least one proximity sensor 166 and at least one target object 167, said proximity sensor 166 cooperating with said target object 167 to detect at least the mutual position, preferably the mutual position and relative orientation, of said first elongated element and said second elongated element. Preferably, one between said first element elongated body and said second element elongated body comprises said proximity sensor and the other comprises said target object.

According to an embodiment, said master input tool 106 comprises at least one joint spring 120 biasing at least said first element cantilevered portion 122 of said first element elongated body 114 away from said second element cantilevered portion 123 of said second element elongated body 116, along said single degree of freedom of motion.

According to an embodiment, said joint spring 120 angularly biases said first element elongated body 114 and said second element elongated body 116 towards said at least one open position.

According to an embodiment, said joint spring 120 is interposed between said first element elongated body 114 and said second element elongated body 116.

According to an embodiment, said joint spring 120 is interposed between said first element joint portion 122 and said second element joint portion 123.

According to an embodiment, said joint spring 120 is a torsional spring.

According to an embodiment, said joint spring 120 is an axial spring.

According to an embodiment, said joint spring 120 is located around said tool joint 118. According to an embodiment, said joint spring 120 is located around a tool joint pin 124 of said tool joint 118.

According to an embodiment, said joint spring 120 exerts an elastic bias action directed to increase the master gripping angle α+γ.

According to an embodiment, each of said first element elongated body 114 and said second element elongated body 116 is made in single piece.

According to an embodiment, each of said first element elongated body 114 and said second element elongated body 116 is made of polymeric material.

According to an embodiment, each of said first element elongated body 114 and said second element elongated body 116 is made by molding, preferably by injection molding. In this way is reduced the number of parts to be assembled together to form said master input tool 106.

According to an embodiment, the angle between said first elongated element of surgical grip device 142 and said second elongated element of surgical grip device 143 is equal to the paired, along a master-slave pair, master gripping angle α+γ.

According to an embodiment, said first elongated element of surgical grip device 142 and said second elongated element of surgical grip device 143 define a slave gripping angle β therebetween. Preferably, said first elongated element of surgical grip device 142 and said second elongated element of surgical grip device 143 of said slave surgical grip device 117 are movable in respect to one another between at least one open position, wherein said slave gripping angle β is greater than a predefined slave grip threshold, and at least one closed position, wherein said slave gripping angle β is lower than said predefined slave grip threshold, preferably substantially equal to zero. Preferably, when said first elongated element of surgical grip device 142 and said second elongated element of surgical grip device 143 are in a closed position, said first elongated element of surgical grip device 142 and said second elongated element of surgical grip device 143 are aligned, preferably along a slave grip device longitudinal axis Y-Y.

According to an embodiment, when said first element elongated body 114 and said second element elongated body 116 are in a closed position, the paired slave surgical grip device 117 is in a closed position.

According to a preferred embodiment, said master input tool 106 comprises a grip force detector device 125, detecting the gripping pressure action 147 exerted by the surgeon's fingers 111, 112 moving said first element elongated body 114 and said second element elongated body 116 close one another other below said predefined grip threshold angle γ.

According to a preferred embodiment, said grip force detector device 125 of said master input tool 106 detects the gripping pressure action 147 when said master gripping angle is lower that said grip threshold angle γ.

According to an embodiment, when the gripping pressure action 147 exerted by the surgeon's fingers 111, 112 moves said first element elongated body 114 and said second element elongated body 116 close one another other below said predefined grip threshold angle γ, determines a paired grip force increase exerted by said surgical grip device 117. In this way, the surgeon is allowed to be aware when the slave surgical grip device 117 is cutting at least a portion of a patient anatomy by mechanical force feedback.

According to an embodiment, said grip force detector device 125 comprise at least one trigger 126 rotatably connected to said first element elongated body 114 forming a trigger joint 127.

According to an embodiment, said trigger joint 127 is a pin joint comprising a trigger pin 164. According to an embodiment, said trigger joint 127 is an hinge.

According to an embodiment, said trigger 126 comprises a trigger root 128 that forms a portion of said trigger joint 127 and a trigger free end 129 extending cantilevered in respect of said trigger joint 127.

According to an embodiment, said grip force detector device 125 comprises at least one grip spring 130 biasing said trigger free end 129 away from said first element elongated body 114, so that said trigger 126 extends cantilevered facing said second element elongated body 116.

According to an embodiment, when said trigger free end 129 is urged towards said first element elongated body 114 by means of the gripping pressure action exerted by the surgeon's fingers 111, 112, said grip spring 130 exerts an elastic return action directed to contrast said gripping pressure action exerted by the surgeon's fingers 111, 112, in such way to make the surgeon aware of said paired grip force increase exerted by said slave grip device 117 by means of mechanical force feedback.

According to an embodiment, said second element elongated body 116 comprises a trigger abutment portion 140, that forms an abutment wall for the trigger free end 129, when said gripping pressure action exerted by the surgeon's fingers 111, 112 moves said first element elongated body 114 and said second element elongated body 116 close one another other below a predefined grip threshold γ.

According to an embodiment, said trigger abutment portion 140 defines a trigger seat 149 for receiving at least a portion of said trigger 126 when said master input tool 106 is in a closed position.

According to an embodiment, said trigger abutment portion 140 defines a trigger seat 149, suitable for receiving at least said trigger free end 129, when said gripping pressure action exerted by the surgeon's fingers 111, 112 moves said first element elongated body 114 and said second element elongated body 116 close one another other below a predefined grip threshold γ.

According to an embodiment, said grip force detector device 125 comprises at least one load cell.

According to an embodiment, said sensing assembly 119 comprises at least one first sensor 134. Preferably, said first sensor 134 is integral with said first elongated element 113, preferably integral with said first element elongated body 114.

According to an embodiment, said first element elongated body 114 delimits at least one first slot 138 receiving at least a portion of said sensing assembly 119. According to an embodiment, said at least one first slot 138 receives at least a portion of said sensing assembly 119 in a detachable manner, so that the master input tool 106 comprising or devoid of said sensing assembly 119 is disposable.

According to an embodiment, said sensing assembly 119 comprises at least one sterile sensor container 165, for example a plastic bag or a plastic box and/or the like, enclosing at least one of said first sensor 134 or said second sensor 135. In this way, sensor assembly 119 sterility is achievable avoiding to require sensor 134, 135 replacement after a single surgery. Thereby, master input tool body 106 may be made disposable and sensors 134, 135 can be utilized multiple times because their sterility is preserved. Preferably, also the wired connections 136, 137 to sensors 134, 135 are enclosed by sterile boxes 165 or appendix thereof.

According to an embodiment, said first slot 138 receives said first sensor 134.

According to an embodiment, said first sensor 134 is operatively connected to said field generator 107 by means of a first sensor connection 136. According to an embodiment, said first sensor connection 136 is a wired connection. According to an embodiment, said first sensor connection 136 is a wireless connection.

According to a preferred embodiment, said sensing assembly 119 comprises at least one second sensor 135. Preferably, said second sensor 135 is integral with said second elongated element 115.

According to an embodiment, said second element elongated body 116 delimits at least one second slot 139 receiving at least a portion of said sensing assembly 119. According to an embodiment, said second slot 139 receives at least a portion of said sensing assembly 119 in a detachable manner, so that the master input tool 106 comprising or devoid of said sensing assembly 119 is disposable.

According to an embodiment, said second slot 139 receives said second sensor 135.

According to an embodiment, said second sensor 135 is operatively connected to said field generator 107 by means of a second sensor connection 137. According to an embodiment, said second sensor connection 137 is a wired connection. According to an embodiment, said second sensor connection 137 a wireless connection.

According to an embodiment, said first slot 138 faces opposite in respect of said second slot 139, so that a unique arrangement of said sensing assembly 119 is allowed. In this way, the chances of misplacing sensing assembly 119 are significantly reduced.

According to an embodiment, said slots 138, 139 comprise at least one flag element, for example a notch and/or the like to signal whether the sensor 134, 135 is operatively received in the respective slot 138. 139.

According to an embodiment, said slots 138, 139 have different flag element to each other so that a sensor 134, 135 can be operatively connected to only one of the slots 138, 139.

According to an embodiment, the arrangement of said slots 138, 139 is asymmetric. According to an embodiment, the arrangement of said sensors 134, 135 is asymmetric.

According to an embodiment, said first slot 138 is opposite to said second slot 139 with respect of said tool longitudinal axis X-X.

According to an embodiment, said slots 138, 139 have substantially the same shape and size.

According to an embodiment, said slots 138, 139 have substantially the shape of a parallelepiped.

According to an embodiment, said slots 138, 139 are provided near the free end portion of each elongated body 114, 116, so that to have the maximum linear displacement keeping constant the angular displacement, and sensors 134, 135 are received in respective slots 138, 139.

According to an embodiment, said slots 138, 139 are provided at maximum distance from the tool joint 118, so that to have the maximum linear displacement keeping constant the angular displacement and sensors 134, 135 are received in respective slots 138, 139.

For example, the slots 138, 139 and sensors 134, 135 are provided near or at the distal end of each elongated body 114, 116, when the tool joint 118 is near or at the proximal end thereof.

According to an embodiment, said first element cantilevered portion 122 and said second element cantilevered portion 123 of said master input tool 106 define a first longitudinal side 150, and wherein a second longitudinal side 151 is defined opposite to said first longitudinal side 150 in respect of said tool joint 118.

According to an embodiment, said first sensor connection 136 and said second sensor connection 137 are both wired connections, and wherein the wires of said first sensor connection 136 and said second sensor connection 137 are both gathered on a same longitudinal side 150;151 of said master input tool 106. In this way the encumber of said sensor connections is reduced.

According to an embodiment, said master input tool 106 comprises at least one back-of-hand resting portion 131, designed to touch at least a portion of the surgeon's back-of-hand 141, when in operative conditions.

According to an embodiment, the position of said manipulandum surface 109, 110 along the tool longitudinal axis X-X is interposed between said first tool longitudinal side 150 and said sensing assembly 119. According to an embodiment, the position of said manipulandum surface 109, 110 along the tool longitudinal axis X-X is interposed between said first tool longitudinal side 150 and said force detector device 125.

According to an embodiment, said first element elongated body 114 comprises at least one manipulandum surface 109,110, said manipulandum surface 109,110 is located along said first elongated element direction X1-X1 between said first element cantilevered portion 122 and said trigger joint 127. According to an embodiment, said first element elongated body 114 comprises at least one manipulandum surface 109, 110 said manipulandum surface 109,110 is located along said first elongated element direction X1-X1 between said first element joint portion 132 and said trigger joint 127.

According to an embodiment, the position of said manipulandum surface 109, 110 along the tool longitudinal axis X-X is interposed between said second tool longitudinal side 151 and said sensing assembly 119. According to an embodiment, the position of said manipulandum surface 109, 110 along the tool longitudinal axis X-X is interposed between said second tool longitudinal side 151 and said force detector device 125. According to an embodiment, said slave robot assembly 103 further comprises at least one surgical arm 152 manipulating said slave surgical instrument 104. According to an embodiment, said salve robot assembly 103 comprises at least one micromanipulator 153 manipulating said slave surgical instrument 104. Preferably, said at least one micromanipulator 153 is directly connected in series to said surgical arm 152 forming a kinematic chain with said surgical arm 152, said micromanipulator 153 manipulating said slave surgical instrument 104. According to an embodiment, at least two micromanipulators 153 are directly connected in series to said surgical arm 152 forming an at least two-branched kinematic chain with said surgical arm 153.

According to an embodiment, said robotic surgery system 101 comprises at least one robot cart 154 comprising at least one cart ground contact unit 155 and a cart handle 156, said cart handle 156 being suitable for moving at least a portion of the robotic surgery system 101, preferably said slave robot assembly 103, at least within the operating arena. Preferably, said robot cart 154 forms a mechanical and structural support, preferably a movable mechanical and structural support, for the slave robot assembly 103.

According to an embodiment, said robot cart 154 is connected to a power supply cable 157.

According to an embodiment, said robot cart 154 comprises said control unit 105. Preferably said control unit 105 is located integral said robot cart 154.

According to an embodiment, said robot cart 154 comprises said filed generator 107.

According to an embodiment, said master controller assembly 102 further comprises at least one surgical chair 158 comprising at least one seating surface 159 for the surgeon to seat thereon during surgery.

According to an embodiment, said surgical chair 158 being mechanically unconstrained from the slave robot assembly 103, so as to prevent the propagation by mechanical contact of vibrational motion from the surgical chair 158 to the slave robot assembly 103. In this way, is reduced the risk of unwanted commands transmittal to the slave surgical robot 103, and particularly to said slave surgical instrument 104.

According to an embodiment, said surgical chair 158 comprises said field generator 107 so that said field volume is integral with at least a portion of said surgical chair 158.

According to an embodiment, said master input tool 106 is operatively connected to said surgical chair 158 by means of a chair operative connection 160. According to an embodiment, said chair operative connection 160 is a wired connection. According to an embodiment, said chair operative connection 160 is a wireless connection.

According to a general embodiment, it is provided a master controller assembly 102 for a robotic surgery system 101, said robotic surgery system 101 further comprising a slave robot assembly 103 comprising a slave surgical instrument 104 having a surgical grip device 117 providing the slave surgical instrument 104 with a grip degree-of-freedom of motion.

Said master controller assembly 102 comprises a master input tool body 106 and a sensing assembly 119, according to any one of the embodiments described above.

According to a preferred embodiment, said master input tool 106 being suitable to be hand-held and manipulated by a surgeon from various locations of an operating arena during surgery, said master input tool 106 being suitable for receiving a manual command.

According to a preferred embodiment, said master input tool 106 comprising at least one manipulandum surface 109, 110, designed to be hand-held by the surgeon's fingers 111, 112.

According to a preferred embodiment, said master input tool 106 is mechanically unconstrained from said slave robot assembly 103, in such way that said master input tool 106 being naturally movable, rotatable and spinnable by a surgeon.

According to a preferred embodiment, said at least one manipulandum surface 109, 110 is a convex surface, so that said master input tool 106 can be rolled between surgeon's fingers 111, 112 around a tool longitudinal axis X-X.

According to a preferred embodiment, said master input tool 106 comprises a first elongated element 113 having an first element elongated body 114, wherein said first element elongated body 114 is a rigid body, and wherein said master input tool 106 comprises a second elongated element 115 having an second element elongated body 116, wherein said second element elongated body 116 is a rigid body.

According to a preferred embodiment, said master input tool 106 comprises a tool joint 118 connecting and articulating said first element elongated body 114 and said second element elongated body 116, providing a single degree of freedom of motion between said first element elongated body 114 and said second element elongated body 116.

According to a preferred embodiment, said master controller assembly 102 comprises at least one sensing assembly 119 detecting at least the mutual position of said first element elongated body 114 and said second element elongated body 116, so that a gripping pressure action exerted by the surgeon's fingers 111, 112 on said master input tool 106 moving said first element elongated body 114 and said second element elongated body 116 close one another other determines a paired grip motion of said surgical grip device 117.

By virtue of the features described above, provided either separately or in combination, where applicable, in particular embodiments, it is possible to satisfy the sometimes contrasting needs disclosed above, and to obtain the aforesaid advantages, and in particular:

it is provided a hand-held ungrounded master controller assembly of simple manufacturing and at the same time capable of accurate and reliable sensing;

it is provided a master controller assembly where no need of structural constrain to the robot is required, and the connection to the robot may be wired for data transmission purposes;

the surgeon is provided with improved freedom of motion and at the same time with a familiar tool for performing robotic surgery;

it is provided a master controller assembly particularly suitable for robotic microsurgery;

the mechanical features of the sensing assembly and of the slots receiving the sensors allow a unique arrangement of the sensor thereby avoiding misplacements resulting in an improved safety in respect of known solutions and at the same time without requiring high costs for manufacture;

the master input tool body may be made disposable and the sensing assembly may be not.

Those skilled in art may make many changes and adaptations to the embodiments described above or may replace elements with others which are functionally equivalent in order to satisfy contingent needs without however departing from the scope of the appended claims.

### LIST OF REFERENCES

101 Robotic surgery system

102 Master controller assembly

103 Slave robot assembly

104 Slave surgical instrument, or surgical instrument

105 Control unit

106 Master input tool body, or master input tool

107 Field generator

109 First manipulandum surface

110 Second manipulandum surface

111 Surgeon finger

112 Further surgeon finger

113 First elongated element

114 First element elongated body, or first elongated element body

115 Second elongated element

116 Second element elongated body, or second elongated element body

117 Surgical grip device, or slave surgical grip device

118 Tool joint

119 Sensing assembly

120 Joint spring

121 Friction enhanced portion

122 First element cantilevered portion

123 Second element cantilevered portion

124 Tool joint pin

125 Grip force detector device

126 Trigger

127 Trigger joint

128 Trigger root

129 Trigger free end

130 Trigger spring

131 Back-of-the-hand resting portion

132 First element joint portion

133 Second element joint portion

134 First sensor

135 Second sensor

136 First sensor connection

137 Second sensor connection

138 First slot

139 Second slot

140 Trigger abutment portion

141 Surgeon's back-of-hand

142 First elongated element of surgical grip device

143 Second elongated element of surgical grip device

144 Surgical grip joint

145 Joint portion of surgical grip device

146 Free end of surgical grip device

147 Gripping pressure action

148 Paired slave grip motion

149 Trigger seat

150 First longitudinal side

151 Second longitudinal side

152 Surgical arm, or slave surgical arm

153 Micromanipulator

154 Robot cart

155 Cart ground contact unit

156 Cart handle

157 Power supply cable

158 Surgical chair

159 Seating surface

160 Chair operative connection

161 Manual command

162 First command signal

163 Second command signal

164 Trigger pin

165 Sterile sensor container

166 Proximity sensor

167 Target object

X-X Tool longitudinal axis

X1-X1 First element direction

X2-X2 Second element direction

Y-Y Slave grip device longitudinal axis

α+γ Master gripping angle

β Slave gripping angle

γ Grip threshold angle

1. A master controller assembly for a robotic surgery system, said robotic surgery system comprising a slave robot assembly comprising a slave surgical instrument having a surgical grip device providing the slave surgical instrument with a grip degree-of-freedom of motion, the master controller assembly comprising:
at least one master input tool to be hand-held and manipulated by a surgeon from various locations of an operating arena during surgery, and
at least one sensing assembly;
wherein:
said at least one master input tool is configured to receive a manual command;
said at least one master input tool comprises at least one manipulandum surface, designed to be hand-held by surgeon's fingers;
said at least one master input tool is mechanically unconstrained from said slave robot assembly, said at least one master input tool being naturally movable, rotatable and spinnable by the surgeon;
said at least one manipulandum surface is a convex surface, so that said at least one master input tool is rollable between the surgeon's fingers around a tool longitudinal axis;
said at least one master input tool further comprises a first elongated element having a first element elongated body, wherein said first element elongated body is a rigid body;
said at least one master input tool further comprises a second elongated element having a second element elongated body, wherein said second element elongated body is a rigid body;
said at least one master input tool further comprises a tool joint connecting and articulating said first element elongated body and said second element elongated body, providing a single degree of freedom of motion between said first element elongated body and said second element elongated body;
said at least one sensing assembly detects at least a mutual position of said first element elongated body and said second element elongated body, so that a gripping pressure action exerted by the surgeon's fingers on said at least one master input tool moving said first element elongated body and said second element elongated body closer to one another determines a paired grip motion of said surgical grip device.

2. The master controller assembly according to claim 1, wherein said first element elongated body delimits a first slot receiving at least one portion of said at least one sensing assembly.

3. The master controller assembly according to claim 1, wherein said at least one sensing assembly further comprises a first sensor; and said first slot receives said first sensor of said sensing assembly so that said first sensor is integral with said first elongated element.

4. The master controller assembly according to claim 2, wherein said first slot receives said at least one portion of said at least one sensing assembly in a detachable manner, so that the at least one master input tool comprising or devoid of said at least one sensing assembly is disposable.

5. The master controller assembly according to claim 3, wherein said first sensor is operatively connected to a master tracking device by a first sensor connection, and wherein said first sensor connection is a wired connection or a wireless connection.

6. The master controller assembly according to claim 2, wherein said second element elongated body delimits a second slot receiving at least one portion of said at least one sensing assembly.

7. The master controller assembly according to claim 6, wherein said at least one sensing assembly further comprises a second sensor; and
said second sensor is integral with said second elongated element; and/or wherein
said second slot receives at least one portion of said at least one sensing assembly
in a detachable manner, so that the at least one master input tool comprising or devoid of said at least one sensing assembly is disposable; and/or wherein
said second slot receives said second sensor; and/or wherein
said second sensor is operatively connected to said master tracking device by a second sensor connection, and wherein said second sensor connection is a wired connection or a wireless connection.

8. The master controller assembly according to claim 6, wherein said first slot faces opposite in respect of said second slot, to form a unique arrangement of said at least one sensing assembly.

9. The master controller assembly according to claim 6, wherein said first slot is opposite to said second slot with respect of said tool longitudinal axis.

10. The master controller assembly according to claim 6, wherein said first and second slots are provided near a free end portion of each of said first element elongated body and second element elongated body, to have a maximum linear displacement keeping constant angular displacement, and the first and second sensors are received in respective first and second slots; and/or wherein
said first and second slots are provided at maximum distance from the tool joint, to have the maximum linear displacement keeping constant the angular displacement and the first and second sensors are received in respective first and second slots; and/or wherein
the first and second slots and the first and second sensors are provided proximate or at a distal end of each of said first element elongated body and second element elongated body, when the tool joint is proximate or at the proximal end thereof.

11. The master controller assembly according to claim 1, wherein said at least one sensing assembly comprises at least one sterile sensor container, enclosing at least one of said first sensor or second sensor, to preserve sterility of the at least one sensing assembly.

12. The master controller assembly according to claim 7, wherein said first sensor connection and said second sensor connection are both wired connections, and wires of said first sensor connection and said second sensor connection are both gathered on a same longitudinal side of said at least one master input tool.

13. The master controller assembly according to claim 6, wherein said first and second slots comprise at least one flag element, including a notch and/or similar elements, to signal whether the first and second sensors are operatively received in respective first and second slots; and/or wherein
said first and second slots have different flag elements so that a sensor is operatively connected to only one of the first and second slots; and/or wherein
arrangement of said first and second slots is asymmetric; and/or wherein
arrangement of said first and second sensors is asymmetric; and/or wherein
said first and second slots have a substantially same shape and size; and/or wherein
said first and second slots have substantially a parallelepiped shape.

14. The master controller assembly according to claim 1, wherein said at least one master input tool comprises a back-of-hand resting portion, designed to touch at least one portion of the surgeon's back-of-hand in operative conditions.

15. The master controller assembly according to claim 1, wherein:
each of said first element elongated body and said second element elongated body is made in single piece; and/or wherein
each of said first element elongated body and said second element elongated body is made of polymeric material; and/or wherein
each of said first element elongated body and said second element elongated body is made by molding.

16. The master controller assembly according to claim 1, wherein:
at least one of said first element elongated body and said second element elongated body comprises said at least one manipulandum surface; and/or wherein
each of said first element elongated body and said second element elongated body comprises said at least one manipulandum surface; and/or wherein
said at least one manipulandum surface comprises a friction enhanced portion, improving gripping of surgeon's fingers; and/or wherein
said at least one manipulandum surface is a portion of a cylindrical surface.

17. The master controller assembly according to claim 1, wherein said tool joint is a hinge providing a single degree of freedom of motion of rotation between said first element elongated body and said second element elongated body, so that said first element elongated body and said second element elongated body are movable in respect of one another of an angular motion; and/or wherein
said tool joint is a pin joint providing a single degree of freedom of motion of rotation between said first element elongated body and said second element elongated body; and/or wherein
the angle between said first element elongated body and said second element elongated body is a master gripping angle; and/or wherein
said first element elongated body and said second element elongated body are movable in respect of one another of angular motion between an open position, wherein said master gripping angle is greater than a predefined grip threshold angle, and a closed position, wherein said master gripping angle is smaller than a predefined grip threshold angle; and/or wherein
said master gripping angle is equal to or lower than 60 degrees, when said first element elongated body and said second element elongated body are in the open position; and/or wherein
said first element elongated body comprises a first element joint portion, forming a portion of said tool joint, and a first element cantilevered portion, located opposite to said first element joint portion along the tool longitudinal axis; and/or wherein
said second element elongated body comprises a second element joint portion, forming a portion of said tool joint, and a second element cantilevered portion, located opposite to said second element joint proximal portion along the tool longitudinal axis; and/or wherein
said first element joint portion and said second element joint portion cooperate to form said tool joint; and/or wherein
a relative spatial position of said first element cantilevered portion and said second element cantilevered portion is rigidly determined by said master gripping angle width; and/or wherein
said at least one master input tool comprises a joint spring biasing at least said first element cantilevered portion of said first element elongated body away from said second element cantilevered portion of said second element elongated body, along said single degree of freedom of motion; and/or wherein
said first element cantilevered portion and said second element cantilevered portion are located at a predefined distance from said tool joint along said first element elongated body and said second element elongated body, respectively; and/or wherein
said joint spring is interposed between said first element elongated body and said second element elongated body; and/or wherein
said joint spring is interposed between said first element joint portion and said second element joint portion; and/or wherein
said joint spring is located around said tool joint; and/or wherein
said joint spring is located around a tool joint pin of said tool joint; and/or wherein
said joint spring exerts an elastic bias action directed to increase the master gripping angle; and/or wherein
said at least one master input tool comprises a grip force detector device, to detect the gripping pressure action exerted by the surgeon's fingers moving said first element elongated body and said second element elongated body close one another other below a predefined grip threshold angle; and/or wherein
when the gripping pressure action exerted by the surgeon's fingers moves said first element elongated body and said second element elongated body closer to one another below said predefined grip threshold angle determines a paired grip force increase exerted by said surgical grip device; and/or wherein
said master controller assembly comprises at least one master tracking device generating a predefined field volume; and/or wherein
said at least one sensing assembly detecting at least the position of said at least one master input tool within said predefined field volume; and/or wherein
said at least one sensing assembly comprises at least one joint sensor, preferably an encoder, located within said tool joint.

18. Robotic A robotic surgery system comprising:
the master controller assembly according to claim 1;
a robot assembly, comprising a slave surgical instrument designed to operate on a patient anatomy, said slave surgical instrument comprising a surgical grip device providing the slave surgical instrument with a grip degree-of-freedom of motion;
a control unit for receiving a first command signal containing information about a manual command and transmitting a second command signal containing information about said manual command to the slave robot assembly to actuate said slave surgical instrument.

19. The robotic surgery system according to claim 18, wherein field generator is a magnetic field generator.
